(19) United States
(12) Patent Application Publication
(10) Pub. No.: US 2010/0204564 A1
(43) Pub. Date: Aug. 12, 2010

(54) AccuPort Locator

(76) Inventors: Walter A. Dobson, (US); Tam Barrington Dobson, (US)

Correspondence Address: Warren Norred, 2707 Yorkfield Court, Arlington, TX, 76001, US

(21) Appl. No.: 12/367,537

(22) Filed: Feb. 8, 2009

(51) Int. Cl. A61B 6/12

(52) U.S. Cl. 600/424

(57) ABSTRACT

To avoid exposing medical personnel to frequent radiation during patient treatment, a radiopaque indicator is used to locate catheters or similar subcutaneous ports and devices. An attached extension rod allows the medical personnel to remain farther outside a fluoroscope's radiated area, but still manipulate the indicator. Because it is radiopaque, personnel can see its position relative to a patient's extra corporal port on a fluoroscope, fix the position precisely, make the puncture and adjustment and then address the patient's medical needs after completing the barium swallow or other medical treatment.

### CROSS-REFERENCE TO RELATED APPLICATIONS

TBD.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO A “SEQUENCE LISTING,” A TABLE, OR A COMPUTER PROGRAM LISTING APPENDIX SUBMITTED ON COMPACT DISC AND AN INCORPORATION-BY-REFERENCE OF THE MATERIAL ON THE COMPACT DISC

None.

### BACKGROUND OF THE INVENTION

1) Field of the Invention

The present invention pertains to a radiopaque indicator used to locate a subcutaneous access or injection port.

2) Description of the Related Art

Subcutaneous ports are known in the medical arts and used for various purposes, including medicine administration and gastric band adjustment by extracorporeal transdermal injection.

These ports are generally located under the skin and are easy to find. However, with gastric banding, ports are usually fastened to the abdominal wall underneath layers of tissue and skin, and deeper in the adipose tissue (fat). Clinicians (e.g., surgeons, physicians, nurses) may have to struggle in order to manipulate and stabilize these ports, which can move around and tilt or flip if not stabilized to the surrounding tissue during the surgical procedure.

As a patient with a gastric band loses weight, the restriction or stoma size will increase in diameter thus allowing the patient to eat more without the feeling of restriction and/or regurgitation. This allows the patient to eat more and gain weight. In this circumstance, the access port must be located, punctured with a sterile saline filled syringe attached to sterile Huber type needle, and adjusted till the stoma is tight enough to restrict food volume intake, but not so tight as to cause vomiting or excessive regurgitation. This port puncture with Huber needle can be difficult particularly if the port is angled or deep.

Due to the difficulty of locating subcutaneous ports used with gastric bands and other similar procedures, clinicians sometimes struggle to locate the center of the port and can accidentally cause damage to the port or puncture the tubing, which may cause the port to flip or the tubing to leak during adjustment procedures. These mistakes can threaten a patient's health, requiring additional surgery to replace the damaged port or tubing, and increase risk of infection, risk of intra corporal puncture with bowel puncture and peritonitis, even risk of death. Dealing with these errors typically results in additional costs to patients and insurance carriers.

Clinicians use a combination of hand manipulation of the patient's abdomen to manipulate the port so that it is flat and accessible. The fluoroscope locates the center of the port, which can expose both patient and medical personnel to additional radiation from the fluoroscope. The port is radiopaque, so personnel can see its location on the fluoroscope monitor, and can then use their hands to stabilize the port and proceed with the procedure while continuously viewing the monitor. With the use of the AccuPort Locator, the port puncture is more accurate, so that the procedure is less likely to result in tubing puncture and port damage with subsequent port leaking, tilting, and/or flipping.

Because patients are not subject to the effects of radiation except during treatment, the health hazard to the patient radiation is minimal, though fluoro time increases when clinicians struggle to locate the port, level a tilted port, or fail to notice a flipped port. However, the hazard to the clinician who performs the procedure many times several days a week is much greater because they expose their hands with each procedure when the fluoroscope is used during port puncture and adjustment.

### BRIEF SUMMARY OF THE INVENTION

The invention is a radiopaque indicator with an extension handle made to assist medical personnel with the following objects:

One object of the invention is to assist medical personnel in locating subcutaneous ports, and once found, to ensure that the port is properly upright (not flipped), and then provide assistance in targeting the center of the port during percutaneous injections and aspirations.

Another object of the invention is to reduce radiation exposure to the patient and clinician during procedures using fluoroscopic controlled punctures by procedure time and fluoro time.

Another object of the invention is to help avoid patient injury, abdominal or organ perforation by visually monitoring the guidance of the needle.

Another object of the invention is to help the clinician avoid injury to the port and connector tubing by avoiding puncture of the silicone connector tubing creating a leak, or flipping the port upside down by too much pressure on a side.

Another object of the invention allows for exact easy of placement and decreased amount of local anesthesia used, if so desired by patient. By using the AccuPort Locator the clinician can transfer the location of the center of the port to the surface of the skin which coincides with the center point of the AccuPort Locator allowing clinician to perform the subcutaneous local injection exactly over the center of the port.

### BRIEF DESCRIPTION OF THE SEVERAL VIEWS OF THE DRAWINGS

FIG. 1 shows a top drawing of the AccuPort Locator, with a locating ring surrounding a locating pointer mounted to the inside of the locating ring, and the locating indicator mounted to one end of an extension rod.

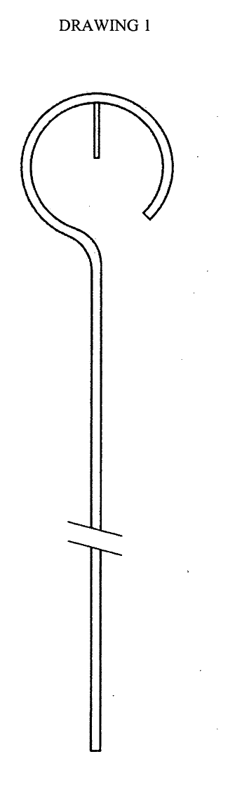

### DETAILED DESCRIPTION OF THE INVENTION

According to the present invention, the foregoing objects and other advantages are attained by mounting a radiopaque indicator to a mounting rod. By using the indicator at a distance, the clinician can avoid an area of concentrated fluoroscopic radiation, but have a clear image on the fluoroscopic monitor to assist in finding the center of the septum safely and puncturing port.

The sterile AccuPort Locator is placed on prepped skin approximately the same shape and size as the port and shows the clinician under fluoroscopic control proper fluoroscopic orientation up or down, left or right prior to accurately locating and positioning the AccuPort Locator over the access port. The center point of the access port is easy to locate after the fluoroscope has been properly oriented because the center corresponds to the center point indicator of the AccuPort Locator. Because the AccuPort Locator is radiopaque, the operator can easily adjust its position accurately on the sterile field of the skin surface using the fluoroscope monitor, then making the needle puncture induction much easier and faster. If the port is so deep that the operator must push it into the skin of an obese patient, the AccuPort Locator will be in the way, and therefore there is an exit channel to allow removal of the AccuPort Locator away from the sterile field and out of the way on the fluoroscopes monitor.

Specifically, the clinician can place the AccuPort Locator on the surface of the patient's surgically prepped skin and view the fluoroscope monitor to determine the relative positioning of the AccuPort indicator in comparison to the center of the subcutaneous port. The clinician can move the collimator around to its proper orientation. Once this orientation is achieved, the center of the indicator can easily be moved over the skin surface until the indicator is centered over the middle of the silicone septum of the port. Once the fluoroscope has been properly orientated and the port has clearly been located, the clinician holds the AccuPort Locator in place with one hand and with the other punctures the skin under direct fluoroscopic control and passes the needle down to the center of the silicone septum and into the port reservoir. The metallic backing of the port will stop the needle when properly placed and the adjustment may be completed. The fluoroscope's C-arm is removed and set up for the barium swallow to check the gastric stoma. The AccuPort Locator is removed from the operative field along with the needle and syringe. Excessive prep solution and/or blood is cleaned off and a Band-Aid placed if needed. A standing barium swallow then follows to finalize that the adjustment is appropriate for the patient.

The essential element of the AccuPort Locator is the indicator element. It is possible that a clinician could use an adhesive-treated radiopaque indicator simply by placing it on a patient's body, turning on the fluoroscope, noting the relative position, turning the fluoroscope off, adjusting the indicator's position on the patient's body, and repeating the process. In this manner, the clinician could avoid fluoroscopic radiation at the cost of the procedure being slower and less accurate.

However, to hasten the process, the AccuPort will generally have an extension rod affixed to the indicator element, allowing the clinician to make adjustments at a distance without repeatedly turning the fluoroscope off and on.

The indicator element needs only to be radiopaque and large enough to be seen on the fluoroscopic monitor. It also should be the same general size and shape of the port in order to fit over its subcutaneous location. The pointing element fixed to the inside diameter such that the pointing element points to the center of the ring element when the device is centered over the patient's subcutaneous port on the patient's skin.

The most common configuration, or best mode, will typically include an extension rod with an indicator affixed to one end, said indicator comprising a radiopaque ring and pointer. The radiopaque ring will have a break in it to allow the clinician to remove the indicator from the field if the port is so deep that pressure on the skin is required to puncture the silicone septum of the port.

Additional configurations could include a vertical extension from any part of the indicator element. A vertical element could be useful in allowing medical personnel to angle the port indicator when under the fluoroscope and match the angle of the indicator to the angle of the subcutaneous port and angle of the C-Arm if the port is positioned inside a patient non-parallel to the patient's skin.

Another configuration could allow the ring itself to be a vertical radiopaque tube instead of a flat ring, which would serve both the locating and angle functions. The medical personnel would then look at the fluoroscope monitor and manipulate the indicator such that the indicator appeared only as a ring on the fluoroscope monitor around the port. Only when the angle of the tube element, the fluoroscope, and port are matched would there be just the ring of the indicator showing on the monitor.

Another configuration would include a marking element on the indicator, so when the location is found, the location would be marked on the patient's body. The indicator ring could have medical ink on one side, so no marking occurred until the medical personnel found the port using the side that has no ink. Once the location is found, however, the indicator would be turned over and pressed on the patient's body to mark the location.

Another configuration would include a small parrel ring attached to the point of the center point of the AccuPort Locator which would allow placement of tattoo ink to be injected exactly in the skin at the center of the ports silicone septum.

Though the best mode includes a handle to manipulate and torque the indicator at a distance and a ring around the indicator, the indicator does not require a handle or the ring around the indicator. The ring is to assist in targeting an area, with the indicator pointing to the middle of the subcutaneous port, but the ring could be of any convenient shape to match the port device.

Another configuration would include a ring attached to the center point of the AccuPort Locator which would allow placement of tattoo ink to be injected exactly at the center of the port's silicone septum in the skin.

There are times when the clinician may need to begin an injection before removing the AccuPort Locator. The AccuPort Locator's typical configuration includes a break in the radiopaque ring element so that the device is removable from the patient once advancement to the puncture site or injection has begun.

1) An apparatus comprising of a radiopaque indicator, with said indicator locating a target area for treatment while viewed under fluoroscopic control with monitor.

2) An apparatus as in claim one with said indicator mounted to an extension rod.

3) An apparatus as in claim one with a surrounding ring or other shape that enables the indicator to be more easily seen on a fluoroscope such that the indicator points to the middle of the ring's indicated area.

4) An apparatus as in claim one with an indicator that has a vertically mounted element added, making the structure three-dimensional so that the apparatus can be more accurately fixed to a patient's target area using a fluoroscope, such as when the port is tilted or adjacent to a large fold of fat

5) An apparatus as in claim one comprising a ring or similar outlining indicator, constructed such that the ring has a break in it, to allow medical personnel to remove the apparatus from the fluoroscopic field if it gets in the way while the targeted area is treated.

6) An apparatus as in claim one comprising a marking element, so that the apparatus leaves a marking, adhesive, or other temporary or permanent coloration on the patient to denote port location after medical personnel cease using the fluoroscope and indicator.

7) A method for locating a subcutaneous port by using a radiopaque indicator to find and provide medical treatment to patients under a fluoroscope, comprising:
a. Placing the patient under a fluoroscope;
b. Setting a radiopaque indictor on the patient's body and using its relative position to the subcutaneous port as viewed in the fluoroscope display, locate the position and angle of a port set inside the patient.

8) The method of claim number 6, comprising the additional step of using an indicator with a handle sufficient to allow the manipulation of the indicator on the patient, while maintaining a distance sufficient to diminish the medical personnel's exposure to undo radiation from the fluoroscope.
